(19) United States
(12) Patent Application Publication
(10) Pub. No.: US 2019/0105070 A1
(43) Pub. Date: Apr. 11, 2019

(54) MEDICAL DEVICE INCLUDING CUTTING MEMBER

(71) Applicant: Lake Region Manufacturing, Inc., Chaska, MN (US)

(72) Inventors: Brent Roeger, Minneapolis, MN (US); Abhishek M. Telang, Minneapolis, MN (US)

(21) Appl. No.: 16/152,448

(22) Filed: Oct. 5, 2018

Related U.S. Application Data
(60) Provisional application No. 62/568,866, filed on Oct. 6, 2017.

(51) Int. Cl. A61B 17/3207; A61L 31/02

(52) U.S. Cl. CPC A61B 17/3207; A61B 2017/320008; A61L 31/022

(57) ABSTRACT

In various examples, a medical cutting device is configured for at least partial insertion within a patient. The medical cutting device includes a flexible elongate member including a distal end and a proximal end. The elongate member including a first material. An intermediate member is attached to the elongate member proximate the distal end of the elongate member. The intermediate member includes a second material different from the first material. A cutting member is attached to and extends outwardly from the intermediate member. The cutting member includes a third material different from the first and second materials, wherein the cutting member is configured to cut an object disposed within the patient. In some examples, the first material of the elongate member includes Nitinol, the second material of the intermediate member includes MP35N, and the third material of the cutting member includes stainless steel.

### CROSS REFERENCE TO RELATED APPLICATIONS

This application claims the benefit of priority to U.S. Provisional Application Ser. No. 62/568,866, filed on Oct. 6, 2017, entitled “NITINOL WIRE TO STAINLESS STEEL SHEET ASSEMBLY WITH CO-ALLOY INTERMEDIATE,” which is incorporated by reference herein in its entirety.

### BACKGROUND

The present invention relates to a medical device, and more specifically relates to a medical device including a cutting member.

A medical device including a cutting member or other material-removal portion that can be navigated into a patient (for instance, through vasculature of the patient) can be useful in one or more of various medical procedures in which material within the patient is to be cut, sliced, abraded, removed, or otherwise separated from other material within the patient. For instance, in some examples, such a device can be used to separate plaque from vessel walls within a patient.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present subject matter can be used to perform a cutting or removal procedure within a patient, such as, for instance, within the vasculature of the patient. In various examples, the present subject matter is advantageous in that it provides an elongate member that is capable of being navigated within the patient (for instance, through vasculature of the patient) to a location within the patient where material is located that is to be cut, abraded, or otherwise removed from within the patient. To that end, the present inventors have recognized the present subject matter can allow for a medical device including the elongate member being at least partially formed from a material that allows navigation through the vasculature of the patient and that is attached to a cutting member that can be formed, sharpened, or otherwise configured to allow for cutting, abrading, or otherwise removing material from within the patient. Also, the present subject matter is advantageous in that it provides for attachment of the elongate member, made from a first material, and the cutting member, made from a second material, even though, in some examples, the first and second materials are difficult to attach to one another. To better illustrate the devices described herein, a non-limiting list of examples is provided here:

Example 1 can include subject matter that can include a medical cutting device configured for at least partial insertion within a patient. The medical cutting device includes a flexible elongate member including a distal end and a proximal end. The elongate member includes a first material. An intermediate member is attached to the elongate member proximate the distal end of the elongate member. The intermediate member includes a second material different from the first material. A cutting member is attached to and extends outwardly from the intermediate member. The cutting member includes a third material different from the first and second materials, wherein the cutting member is configured to cut an object disposed within the patient.

In Example 2, the subject matter of Example 1 is optionally configured such that the first material of the elongate member includes a titanium alloy.

In Example 3, the subject matter of any one of Examples 1 and 2 is optionally configured such that the first material of the elongate member includes Nitinol.

In Example 4, the subject matter of any one of Examples 1-3 is optionally configured such that the second material of the intermediate member includes a cobalt alloy.

In Example 5, the subject matter of any one of Examples 1-4 is optionally configured such that the second material of the intermediate member includes MP35N.

In Example 6, the subject matter of any one of Examples 1-5 is optionally configured such that the third material of the cutting member includes a ferrous material.

In Example 7, the subject matter of any one of Examples 1-6 is optionally configured such that the third material of the cutting member includes stainless steel.

In Example 8, the subject matter of any one of Examples 1-7 is optionally configured such that the elongate member includes at least one of a wire, a hypotube, a cable, a braid, and a coil.

In Example 9, the subject matter of any one of Examples 1-8 is optionally configured such that the intermediate member includes at least one of a wire, a hypotube, a cable, a braid, a sheet, a block, and a coil.

In Example 10, the subject matter of Example 9 is optionally configured such that the intermediate member includes the hypotube disposed at least partially around the distal end of the elongate member.

In Example 11, the subject matter of any one of Examples 1-10 is optionally configured such that the intermediate member is welded to the elongate member.

In Example 12, the subject matter of Example 11 is optionally configured such that the intermediate member is laser welded to the elongate member.

In Example 13, the subject matter of any one of Examples 1-12 is optionally configured such that the cutting member includes a sharpened edge configured to slice through the object disposed within the patient.

In Example 14, the subject matter of any one of Examples 1-13 is optionally configured such that the cutting member includes an abrading surface configured to scrape away a surface of the object disposed within the patient.

In Example 15, the subject matter of any one of Examples 1-14 is optionally configured such that the cutting member includes a rectangular shape.

In Example 16, the subject matter of any one of Examples 1-15 is optionally configured such that the cutting member includes a triangular shape.

In Example 17, the subject matter of any one of Examples 1-16 is optionally configured such that the cutting member includes a rounded shape.

In Example 18, the subject matter of any one of Examples 1-17 is optionally configured such that the cutting member includes a sawtooth shape.

In Example 19, the subject matter of any one of Examples 1-18 optionally includes more than one cutting member extending outwardly from the elongate member.

In Example 20, the subject matter of Example 19 is optionally configured such that the cutting members are substantially evenly spaced from one another.

In Example 21, the subject matter of Example 19 is optionally configured such that the cutting members are unevenly spaced from one another.

In Example 22, the subject matter of any one of Examples 1-21 is optionally configured such that the cutting member is welded to the intermediate member.

In Example 23, the subject matter of Example 22 is optionally configured such that the cutting member is laser welded to the intermediate member.

In Example 24, the subject matter of any one of Examples 1-23 is optionally configured such that the intermediate member includes a slot, at least a portion of the cutting member being disposed and attached within the slot.

In Example 25, the subject matter of Example 24 is optionally configured such that the cutting member includes a tab sized and shaped to fit within the slot, wherein the tab is disposed within the slot.

Example 26 can include, or can optionally be combined with any one of Examples 1-25 to include subject matter that can include a medical cutting device configured for at least partial insertion within a patient. The medical cutting device includes a flexible elongate member including a distal end and a proximal end. The elongate member includes a titanium alloy. An intermediate member is welded to the elongate member proximate the distal end of the elongate member. The intermediate member includes a cobalt alloy. A cutting member is welded to and extends outwardly from the intermediate member. The cutting member includes a ferrous material, wherein the cutting member is configured to cut an object disposed within the patient.

In Example 27, the subject matter of Example 26 is optionally configured such that the titanium alloy of the elongate member includes Nitinol.

In Example 28, the subject matter of any one of Examples 26 and 27 is optionally configured such that the cobalt alloy of the intermediate member includes MP35N.

In Example 29, the subject matter of any one of Examples 26-28 is optionally configured such that the ferrous material of the cutting member includes stainless steel.

In Example 30, the subject matter of any one of Examples 26-29 is optionally configured such that the elongate member includes at least one of a wire, a hypotube, a cable, a braid, and a coil.

In Example 31, the subject matter of any one of Examples 26-30 is optionally configured such that the intermediate member includes at least one of a wire, a hypotube, a cable, a braid, a sheet, a block, and a coil.

In Example 32, the subject matter of Example 31 is optionally configured such that the intermediate member includes the hypotube disposed at least partially around the distal end of the elongate member.

In Example 33, the subject matter of any one of Examples 26-32 is optionally configured such that the intermediate member is laser welded to the elongate member.

In Example 34, the subject matter of any one of Examples 26-33 is optionally configured such that the cutting member includes a sharpened edge configured to slice through the object disposed within the patient.

In Example 35, the subject matter of any one of Examples 26-34 is optionally configured such that the cutting member includes an abrading surface configured to scrape away a surface of the object disposed within the patient.

In Example 36, the subject matter of any one of Examples 26-35 optionally includes more than one cutting member extending outwardly from the elongate member.

In Example 37, the subject matter of any one of Examples 26-36 is optionally configured such that the cutting member is laser welded to the intermediate member.

In Example 38, the subject matter of any one of Examples 26-37 is optionally configured such that the intermediate member includes a slot. At least a portion of the cutting member is disposed and attached within the slot.

In Example 39, the subject matter of Example 38 is optionally configured such that the cutting member includes a tab sized and shaped to fit within the slot, wherein the tab is disposed within the slot.

Example 40 can include, or can optionally be combined with any one of Examples 1-39 to include subject matter that can include a medical cutting device configured for at least partial insertion within a patient. A flexible elongate member includes a distal end and a proximal end. The elongate member includes Nitinol. An intermediate member is laser welded to the elongate member proximate the distal end of the elongate member. The intermediate member includes MP35N. A cutting member is laser welded to and extends outwardly from the intermediate member. The cutting member includes stainless steel, wherein the cutting member is configured to cut an object disposed within the patient.

In Example 41, the subject matter of Example 40 is optionally configured such that the elongate member includes at least one of a wire, a hypotube, a cable, a braid, and a coil.

In Example 42, the subject matter of any one of Examples 40 and 41 is optionally configured such that the intermediate member includes at least one of a wire, a hypotube, a cable, a braid, a sheet, a block, and a coil.

In Example 43, the subject matter of Example 42 is optionally configured such that the intermediate member includes the hypotube disposed at least partially around the distal end of the elongate member.

In Example 44, the subject matter of any one of Examples 40-43 is optionally configured such that the cutting member includes a sharpened edge configured to slice through the object disposed within the patient.

In Example 45, the subject matter of any one of Examples 40-44 is optionally configured such that the cutting member includes an abrading surface configured to scrape away a surface of the object disposed within the patient.

In Example 46, the subject matter of any one of Examples 40-45 optionally includes more than one cutting member extending outwardly from the elongate member.

In Example 47, the subject matter of any one of Examples 40-46 is optionally configured such that the intermediate member includes a slot. At least a portion of the cutting member is disposed and attached within the slot.

In Example 48, the subject matter of Example 47 is optionally configured such that the cutting member includes a tab sized and shaped to fit within the slot, wherein the tab is disposed within the slot.

### BRIEF DESCRIPTION OF THE DRAWINGS

FIG. 1 is a side diagrammatic view of a medical cutting device in accordance with at least one example of the invention.

FIG. 2 is a perspective view of a medical cutting device in accordance with at least one example of the invention.

FIG. 3 is a cross-sectional view of the medical cutting device of FIG. 2 taken along line 3-3 of FIG. 2.

FIG. 4 is a perspective view of a medical cutting device in accordance with at least one example of the invention.

FIG. 5 is a perspective view of a distal end of the medical cutting device of FIG. 4.

FIG. 6 is a perspective view of an intermediate member of the medical cutting device of FIG. 4.

FIG. 7 is a perspective view of a cutting member of the medical cutting device of FIG. 4.

FIG. 8 is a perspective view of a medical cutting device in accordance with at least one example of the invention.

FIG. 9 is a perspective view of a medical cutting device in accordance with at least one example of the invention.

FIG. 10 is a perspective view of a medical cutting device in accordance with at least one example of the invention.

FIG. 11 is a perspective view of a medical cutting device in accordance with at least one example of the invention.

### DETAILED DESCRIPTION

The present patent application relates to a medical device, and more specifically relates to a medical device including a cutting member. In some examples, a medical device includes a cutting member configured to cut, slice, remove, abrade, or otherwise separate an object within a patient. In various examples, as described herein, the medical cutting device can include a cutting member attached to a flexible elongate member to allow for material cutting, abrading, and/or removal within a patient. In some examples, the medical cutting device is configured to be navigated through the vasculature of the patient in order to reach a location within the patient for which material removal is desired.

In some examples, a welding laser is used to join a Nitinol wire, hypotube, slotted hypotube, or cable to a stainless steel (including austenitic, martensitic, or precipitation hardenable alloys) sheet using a MP35N/Co-based alloy intermediate component including a hypotube, sheet, wire, coil, cable, or other similar forms. Both Nitinol and stainless steel are biocompatible materials, and the intermediate MP35N/Co-based alloy component is also biocompatible. The Nitinol component can provide access through tortuous paths due to its super-elastic properties. That is, the Nitinol component allows for flexibility and pushability through tortuous paths due to its super-elasticity. The stainless steel sheet can be a Fe-based alloy that is sharpened to a knife blade finish. In some examples, multiple stainless steel sheets can be attached to the same MP35N/Co-based alloy tube or different MP35N/Co-based alloy tubes to join them to the Nitinol wire. In some examples, the MP35N/Co-based alloy component connects the Nitinol component to the stainless steel/Fe-based alloy component because of difficulties involved with creating a strong joint between Nitinol and stainless steel. By forming two separate joints, a first joint between the Nitinol component and the MP35N/Co-based alloy component and a second joint between the MP35N/Co-based alloy component and the stainless steel/Fe-based alloy component, the Nitinol component can be reliably attached to the stainless steel/Fe-based alloy component. Such a configuration can improve assembly strength by avoiding the mixing of titanium and iron, which forms brittle intermetallics when welded that can compromise weld strength.

In various examples, the present subject matter can be used for one or more of various procedures for removing a material, an object, or tissue from within a patient. In some examples, the present subject matter can be used for an atherectomy procedure to remove material, such as plaque from within a vessel, for instance, to treat atherosclerosis in a patient. The Nitinol wire can be used to reach arteries or veins with the stainless steel/Fe-based sheet molded/shaped in a suitable form to remove plaque. In various examples, the stainless steel/Fe-based sheet can be a blade or can be used as a substrate to deposit abrasive material.

In some examples, the present subject matter can be used for minimally invasive surgery. One or more blades or some other form of grinding or abrading tool can be attached to a distal end of the device, depending on the application. For instance, the present subject matter can be used for an arthroscopic application delivering a stainless steel cutting blade attached to a Nitinol wire, coil, or other form through a tortuous path in a surgery. Nitinol is super-elastic, and this property can be used to facilitate maneuverability of a device around a corner.

Intermediate materials, such as nickel, copper, or other alloys, have been used to join Nitinol to stainless steel/Fe-based alloys. However, nickel, copper, or their alloys are relatively soft and could encounter biocompatibility issues for short or long term use in clinical applications. Such intermediate materials have been used, in some instances, to join wires for guidewire applications.

In some examples, a Nitinol component can be coupled to a stainless steel/Fe-based alloy component using crimping or some other mechanical joint. Soldering or gluing can also be used, in some examples, but may provide limited strength. In some instances, mechanical joints may provide limited tensile strength. In contrast, in some examples, laser welding MP35N to Nitinol exceeded an upper plateau strength of the Nitinol wire.

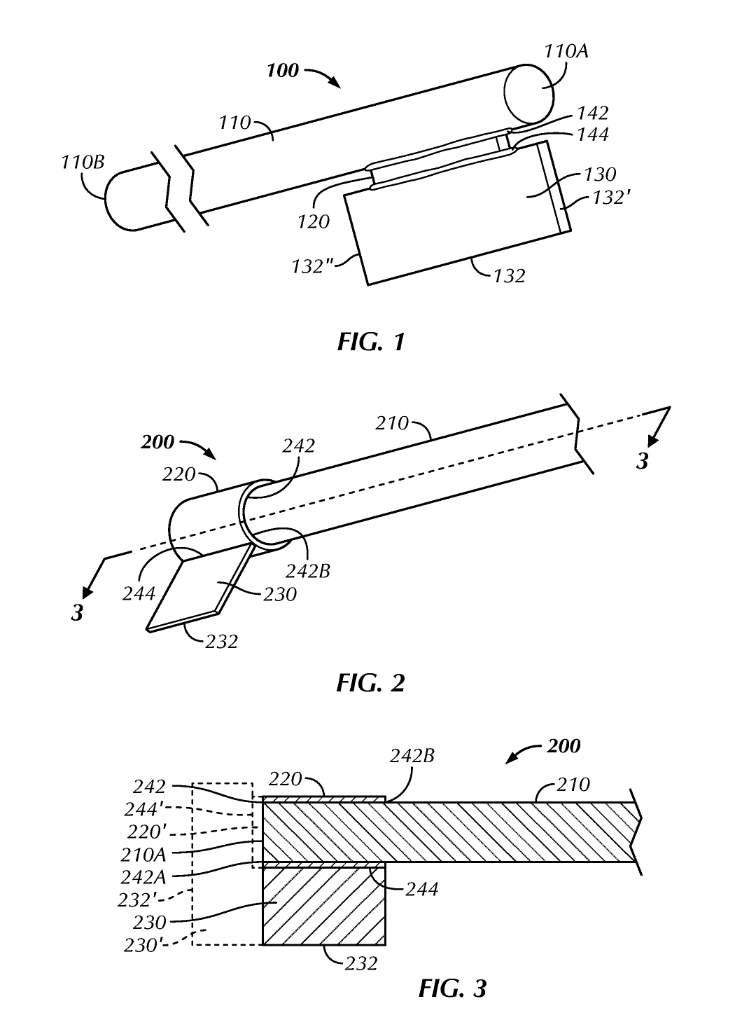

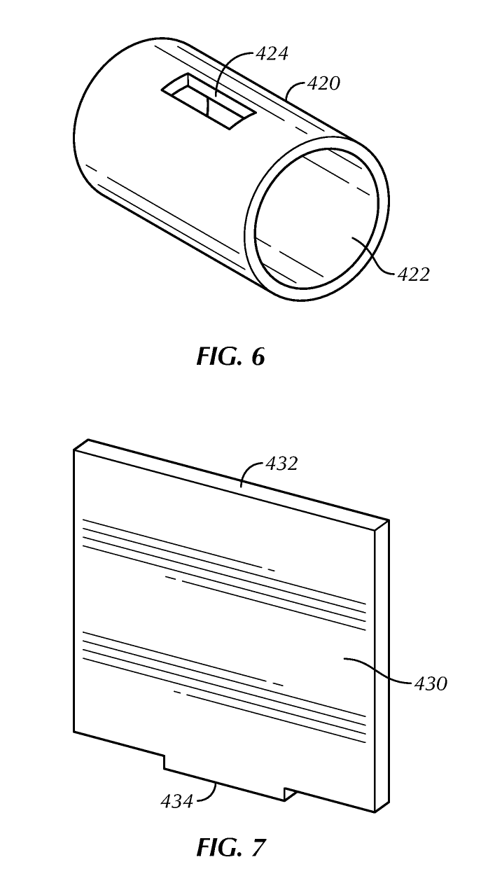

Referring to FIGS. 1-11, various examples of a medical cutting device 100, 200, 400, 800, 900, 1000, 1100 are configured for at least partial insertion within a patient for cutting, slicing, abrading, removing, or otherwise separating at least a portion of material, an object, or tissue within the patient. In various examples, the medical cutting device 100, 200, 400, 800, 900, 1000, 1100 includes a flexible elongate member 110, 210, 410, 810, 910, 1010, 1110 including a distal end 110A, 210A, 410A, 810A, 910A, 1010A, 1110A and a proximal end 110B. In some examples, the flexible elongate member 110, 210, 410, 810, 910, 1010, 1110 is configured to be sufficiently flexible to allow for maneuvering of the flexible elongate member 110, 210, 410, 810, 910, 1010, 1110 along tortuous paths (for instance, through vasculature of the patient), thereby allowing for the flexible elongate member 110, 210, 410, 810, 910, 1010, 1110 to access a location within the patient for a material cutting, slicing, abrading, removing, or separating procedure. In some examples, the elongate member 110, 210, 410, 810, 910, 1010, 1110 includes a first material. In some examples, the first material of the elongate member 110, 210, 410, 810, 910, 1010, 1110 includes a titanium alloy. In further examples, the first material of the elongate member 110, 210, 410, 810, 910, 1010, 1110 includes Nitinol. Although portrayed as a wire in the figures, it should be understood that the elongate member 110, 210, 410, 810, 910, 1010, 1110, in various examples, can include a wire, a hypotube, a cable, a braid, a coil, or a combination thereof.

In some examples, an intermediate member 120, 220, 420, 820, 920, 1020, 1120 is attached to the elongate member 110, 210, 410, 810, 910, 1010, 1110 proximate the distal end 110A, 210A, 410A, 810A, 910A, 1010A, 1110A of the elongate member 110, 210, 410, 810, 910, 1010, 1110. In some examples, the intermediate member 120, 220, 420, 820, 920, 1020, 1120 is welded to the elongate member 110, 210, 410, 810, 910, 1010, 1110. In further examples, the intermediate member 120, 220, 420, 820, 920, 1020, 1120 is laser welded to the elongate member 110, 210, 410, 810, 910, 1010, 1110. In other examples, the intermediate member is attached to the elongate member in other ways, including, for instance, resistance welding, ultrasonic welding, brazing soldering, crimping, using an adhesive, or the like. In some examples, the intermediate member 820, 920 can be spaced a distance proximally from the distal end 810A, 910A. It is noted that although the intermediate members 120, 220, 420, 1020, 1120 are all shown at the respective distal ends 110A, 210A, 410A, 1010A, 1110A, it is contemplated that the intermediate members 120, 220, 420, 1020, 1120 can be spaced proximally from the respective distal ends 110A, 210A, 410A, 1010A, 1110A of the elongate members 110, 210, 410, 1010, 1110. It is further noted that although the intermediate members 820, 920 are each shown spaced proximally from the respective distal ends 810A, 910A, it is contemplated that the intermediate members 820, 920 can be disposed at the respective distal ends 810A, 910A of the elongate members 810, 910.

In some examples, the medical cutting device includes two or more intermediate members spaced along the elongate member. In such examples, a first intermediate member can be at the distal end of the elongate member or spaced proximally from the distal end, and one or more additional intermediate members can be disposed between the first intermediate member and the proximal end of the elongate member. In some examples, the intermediate member 120, 220, 420, 820, 920, 1020, 1120 includes a second material different from the first material. In some examples, the second material of the intermediate member 120, 220, 420, 820, 920, 1020, 1120 includes a cobalt alloy. In further examples, the second material of the intermediate member 120, 220, 420, 820, 920, 1020, 1120 includes MP35N. In various examples, the intermediate member 120, 220, 420, 820, 920, 1020, 1120 includes at least one of a wire, a hypotube, a plug, a bored hypotube, a cable, a braid, a sheet, a block, and a coil.

In some examples, a cutting member 130, 230, 230′, 430, 830, 930, 1030, 1130 is attached to and extends outwardly from the intermediate member 120, 220, 420, 820, 920, 1020, 1120. In some examples, the cutting member 130, 230, 230′, 430, 830, 930, 1030, 1130 is welded to the intermediate member 120, 220, 420, 820, 920, 1020, 1120. In further examples, the cutting member 130, 230, 230′, 430, 830, 930, 1030, 1130 is laser welded to the intermediate member 120, 220, 420, 820, 920, 1020, 1120. In other examples, the cutting member is attached to the intermediate member in other ways, including, for instance, resistance welding, ultrasonic welding, brazing soldering, crimping, using an adhesive, or the like. In some examples, the cutting member 130, 230, 230′, 430, 830, 930, 1030, 1130 is configured to cut an object disposed within the patient. In various examples, the term “cut,” as used herein, includes various forms of separating a material from another material or separating at least a portion of a material from the rest of the material, including, but not limited to, cutting, slicing, scraping, abrading, removing, creating an incision, shaving, trimming, or the like. In some examples, the cutting member 130, 230, 230′, 430, 830, 930, 1030, 1130 includes a third material different from the first and second materials. In some examples, the third material of the cutting member 130, 230, 230′, 430, 830, 930, 1030, 1130 includes a ferrous material. In further examples, the third material of the cutting member 130, 230, 230′, 430, 830, 930, 1030, 1130 includes stainless steel. Although shown in the figures as substantially rectangular in shape, the cutting member 130, 230, 230′, 430, 830, 930, 1030 is not intended to be so limited. As such, in various examples, the cutting member 130, 230, 230′, 430, 830, 930, 1030 can include one or more shapes other than rectangular, such as, but not limited to, a triangular shape, a rounded shape, and a sawtooth shape. In some examples, the cutting member 130, 230, 230′, 430, 830, 930, 1030 includes a sharpened edge 132, 132′, 132″, 232, 232′, 432, 832, 932, 1032 configured to slice through the object disposed within the patient. In some examples, the third material of the cutting member 130, 230, 230′, 430, 830, 930, 1030 is formed from a material capable of being sufficiently sharpened to create the sharpened edge 132, 132′, 132″, 232, 232′, 432, 832, 932, 1032. In other examples, the cutting member 1130 includes an abrading surface 1132 configured to scrape away a surface of the object disposed within the patient. In some examples, the abrading surface 1132 can include, for instance, teeth, holes, grit, protrusions, scoops, or a combination thereof configured to abrade, wear down, or otherwise remove at least a portion of the material, tissue, or other object within the patient.

In this way, in some examples, the medical cutting device 100, 200, 400, 800, 900, 1000, 1100 includes the flexible elongate member 110, 210, 410, 810, 910, 1010, 1110 for maneuvering the medical cutting device 100, 200, 400, 800, 900, 1000, 1100, for instance, within a patient, and the cutting member 130, 230, 430, 830, 930, 1030, 1130 for cutting an object within the patient. The configurations of the examples of the medical cutting device 100, 200, 400, 800, 900, 1000, 1100 described herein allow for the materials of the flexible elongate member 110, 210, 410, 810, 910, 1010, 1110 and the cutting member 130, 230, 230′, 430, 830, 930, 1030, 1130 to be chosen without regard to welding compatibility.

In use, in some examples, the medical cutting device 100, 200, 400, 800, 900, 1000, 1100 is at least partially inserted within a patient such that the cutting member 130, 230, 230′, 430, 830, 930, 1030, 1130 is located at a position within the patient for removal of at least a portion of a material, tissue, or other object within the patient. Once located at the desired location within the patient, in some examples, the medical cutting device 100, 200, 400, 800, 900, 1000, 1100 can be moved with respect to the material, tissue, or other object within the patient to cut the material, tissue, or other object within the patient. In some examples, the medical cutting device 100, 200, 400, 800, 900, 1000, 1100 can be rotated substantially about a longitudinal axis of the elongate member 110, 210, 410, 810, 910, 1010, 1110 of the medical cutting device 100, 200, 400, 800, 900, 1000, 1100 to cut the material, tissue, or other object within the patient with the cutting member 130, 230, 230′, 430, 830, 930, 1030, 1130. In other examples, the medical cutting device 100, 200, 400, 800, 900, 1000, 1100 can be reciprocated substantially along the longitudinal axis of the elongate member 110, 210, 410, 810, 910, 1010, 1110 of the medical cutting device 100, 200, 400, 800, 900, 1000, 1100 to cut the material, tissue, or other object within the patient with the cutting member 130, 230, 230′, 430, 830, 930, 1030, 1130. In still other examples, the medical cutting device 100, 200, 400, 800, 900, 1000, 1100 can be oscillated substantially radially with respect to the elongate member 110, 210, 410, 810, 910, 1010, 1110 of the medical cutting device 100, 200, 400, 800, 900, 1000, 1100 to cut the material, tissue, or other object within the patient with the cutting member 130, 230, 230′, 430, 830, 930, 1030, 1130. In further examples, the medical cutting device 100, 200, 400, 800, 900, 1000, 1100 can be moved in a combination of at least two or rotation, reciprocation, and oscillation to cut the material, tissue, or other object within the patient with the cutting member 130, 230, 230′, 430, 830, 930, 1030, 1130.

Referring now to FIG. 1, in some examples, the medical cutting device 100 includes the intermediate member 120 disposed proximate the distal end 110A of the elongate member 110. In some examples, the intermediate member 120 includes a block or other piece of material disposed between the elongate member 110 and the cutting member 130. That is, the intermediate member 120 of the medical cutting device 100 is disposed only around a portion of a circumference of the elongate member 110, rather than around the entire circumference, as is shown and described herein in other examples. Although shown as a substantially rectangular block, the intermediate member 120, in other examples, can take different forms, such as, for instance, a semi-tubular piece that extends partially around the elongate member 110.

In some examples, the intermediate member 120 is attached to the elongate member 110 at a first joint 142. In further examples, the intermediate member 120 is welded to the elongate member 110 at the first joint 142. In further examples, the intermediate member 120 is laser welded to the elongate member 110 at the first joint 142. In some examples, the first material of the elongate member 110 is compatible with the second material of the intermediate member 120 so as to create a relatively strong weld at the first joint 142. As described above, in some examples, the first material includes a titanium alloy and the second material includes a cobalt alloy, which are compatible materials for forming a robust weld. In some examples, the first joint 142 is formed at least partially along an abutment line between the elongate member 110 and the intermediate member 120. In further examples, the first joint 142 is formed along an entirety of the abutment line between the elongate member 110 and the intermediate member 120.

In some examples, the cutting member 130 is attached to the intermediate member 120 at a second joint 144. In further examples, the cutting member 130 is welded to the intermediate member 120 at the second joint 144. In further examples, the cutting member 130 is laser welded to the intermediate member 120 at the second joint 144. In some examples, the third material of the cutting member 130 is compatible with the second material of the intermediate member 120 so as to create a relatively strong weld at the second joint 144. As described above, in some examples, the third material includes a ferrous alloy and the second material includes a cobalt alloy, which are compatible materials for forming a robust weld. In some examples, the second joint 144 is formed at least partially along an abutment line between the cutting member 130 and the intermediate member 120. In further examples, the second joint 144 is formed along an entirety of the abutment line between the cutting member 130 and the intermediate member 120.

The cutting member 130, in some examples, includes a rectangular sheet extending outwardly from the intermediate member 120. In some examples, the cutting member 130 includes the sharpened edge 132 facing substantially sideways or radially with respect to the medical cutting device 100. In some examples, the cutting member 130 includes the distal sharpened edge 132′ facing substantially distally with respect to the medical cutting device 100 instead of the sharpened edge 132. In other examples, the cutting member 130 includes the proximal sharpened edge 132″ facing substantially proximally with respect to the medical cutting device 100 instead of the sharpened edge 132. In still other examples, the cutting member 130 includes a combination of two or more of the sharpened edge 132, the distal sharpened edge 132′, and the proximal sharpened edge 132″.

In other examples, the cutting member can include a shape other than rectangular, as described above, with one or more sharpened edges, for instance, if a different shape would be more conducive to performing in a particular procedure. In some examples, the cutting member 130 extends from the intermediate member 120 in a radially outward direction from the elongate member 110. In other examples, the cutting member can extend outwardly from the intermediate member at an angle with a tangential component to the elongate member. In some examples, the cutting member 130 can be configured to fold down and/or at least partially wrap around the elongate member 110, for instance, to allow for the medical cutting device 100 to be loaded into and/or passed through a catheter, an introducer, or some other delivery device. In some examples, the cutting member 130 extends substantially longitudinally with respect to the elongate member 110. In other examples, the cutting member 130 can extends along a length of the elongate member 110 angled from longitudinal (for instance, at least partially spirally along the elongate member 110). In further examples, the cutting member 130 can extend from the intermediate member 120 at least partially circumferentially around the elongate member 110. In some examples, at least a portion of the cutting member 130 extends distally with respect to the elongate member 110. In further examples, the cutting member 130 at least partially extends distally away from the distal end 110A of the elongate member 110, in a manner similar to that which is described below with respect to the medical cutting device 200, so as to precede the elongate member 110 and allow for the medical cutting device 100 to cut an object disposed immediately in front of the medical cutting device 100 in addition to or instead of cutting to the side of the medical cutting device 100.

Referring now to FIGS. 2 and 3, the medical cutting device 200, in various examples, is similar in many respects to the medical cutting device 100 described herein. For instance, in some examples, the medical cutting device 200 includes the intermediate member 220 disposed proximate the distal end 210A of the elongate member 210. However, in some examples, the intermediate member 220 of the medical cutting device 200 includes a hypotube instead of the block described with respect to the medical cutting device 100. That is, in some examples, the intermediate member 220 is a hypotube that extends around and is attached to the elongate member 210.

In some examples, the intermediate member 220 is attached to the elongate member 210 at a first joint 242. In further examples, the intermediate member 220 is welded to the elongate member 210 at the first joint 242. In further examples, the intermediate member 220 is laser welded to the elongate member 210 at the first joint 242. In some examples, the first material of the elongate member 210 is compatible with the second material of the intermediate member 220 so as to create a relatively strong weld at the first joint 242. As described above, in some examples, the first material includes a titanium alloy and the second material includes a cobalt alloy, which are compatible materials for forming a robust weld. In some examples, the first joint 242 is formed at least partially around a substantially circumferential abutment line 242A, 242B between the elongate member 210 and the intermediate member 220. In further examples, the first joint 242 is formed along an entirety of the circumferential abutment line 242A, 242B between the elongate member 210 and the intermediate member 220. In various examples, the first joint 242 can be formed at least partially along a distal circumferential abutment line 242A, at least partially along a proximal circumferential abutment line 242B, or at least partially along both the distal circumferential abutment line 242A and the proximal circumferential abutment line 242B.

In some examples, the cutting member 230 is attached to the intermediate member 220 at a second joint 244. In further examples, the cutting member 230 is welded to the intermediate member 220 at the second joint 244. In further examples, the cutting member 230 is laser welded to the intermediate member 220 at the second joint 244. In some examples, the third material of the cutting member 230 is compatible with the second material of the intermediate member 220 so as to create a relatively strong weld at the second joint 244. As described above, in some examples, the third material includes a ferrous alloy and the second material includes a cobalt alloy, which are compatible materials for forming a robust weld. In some examples, the second joint 244 is formed at least partially along an abutment line between the cutting member 230 and the intermediate member 220. In further examples, the second joint 244 is formed along an entirety of the abutment line between the cutting member 230 and the intermediate member 220. In some examples, the intermediate member 220 includes a hypotube with an exterior surface, wherein the cutting member 230 abuts the exterior surface of the intermediate member 220 for attachment to the intermediate member 220 at the second joint 244.

The cutting member 230, in some examples, includes a rectangular sheet extending outwardly from the intermediate member 220. In some examples, the cutting member 230 includes the sharpened edge 232 facing substantially sideways or radially with respect to the medical cutting device 200. In some examples, the cutting member 230 includes a distal sharpened edge (for instance, similar to the distal sharpened edge 132′ of the medical cutting device 100) facing substantially distally with respect to the medical cutting device 200 instead of the sharpened edge 232. In other examples, the cutting member 230 includes a proximal sharpened edge (for instance, similar to the proximal sharpened edge 132″ of the medical cutting device 100) facing substantially proximally with respect to the medical cutting device 200 instead of the sharpened edge 232. In still other examples, the cutting member 230 includes a combination of two or more of the sharpened edge 232, the distal sharpened edge, and the proximal sharpened edge.

In other examples, the cutting member can include a shape other than rectangular, as described above, with one or more sharpened edges, for instance, if a different shape would be more conducive to performing in a particular procedure. In some examples, the cutting member 230 extends from the intermediate member 220 in a radially outward direction from the elongate member 210. In other examples, the cutting member can extend outwardly from the intermediate member at an angle with a tangential component to the elongate member. In some examples, the cutting member 230 can be configured to fold down and/or at least partially wrap around the elongate member 210, for instance, to allow for the medical cutting device 200 to be loaded into and/or passed through a catheter, an introducer, or some other delivery device. In some examples, the cutting member 230 extends substantially longitudinally with respect to the elongate member 210. In other examples, the cutting member 230 can extends along a length of the elongate member 210 angled from longitudinal (for instance, at least partially spirally along the elongate member 210). In further examples, the cutting member 230 can extend from the intermediate member 220 at least partially circumferentially around the elongate member 210. In some examples, at least a portion of the cutting member 230 extends distally with respect to the elongate member 210. In further examples, a portion 230′ (see FIG. 3) of the cutting member 230 at least partially extends distally away from the distal end 210A of the elongate member 210 so as to precede the elongate member 210 and allow for the medical cutting device 200 to cut an object disposed immediately in front of the medical cutting device 200 in addition to or instead of cutting to the side of the medical cutting device 200. In some examples, the portion 230′ of the cutting member 230 includes a distal sharpened edge 232′ either in addition to or instead of the sharpened edge 232 of the cutting member 230. In some examples, the intermediate member 220 includes a distal portion 220′ that extends at least partially over the distal end 210A of the elongate member 210 to allow attachment of the portion 230′ of the cutting member 230 to the distal portion 220′ at a distal joint 244′. In some examples, the intermediate member 220 includes a bored hypotube or a plug having a distal portion 220′ that can be disposed over the distal end 210A of the elongate member 210. In other examples, the portion 230′ of the cutting member 230 can remain unattached to the distal portion 220′. That is, the portion 230′ of the cutting member 230 can cantilever distally from the cutting member 230, thereby relying only on the second joint 244 for attachment of the cutting member 230 to the intermediate member 220.

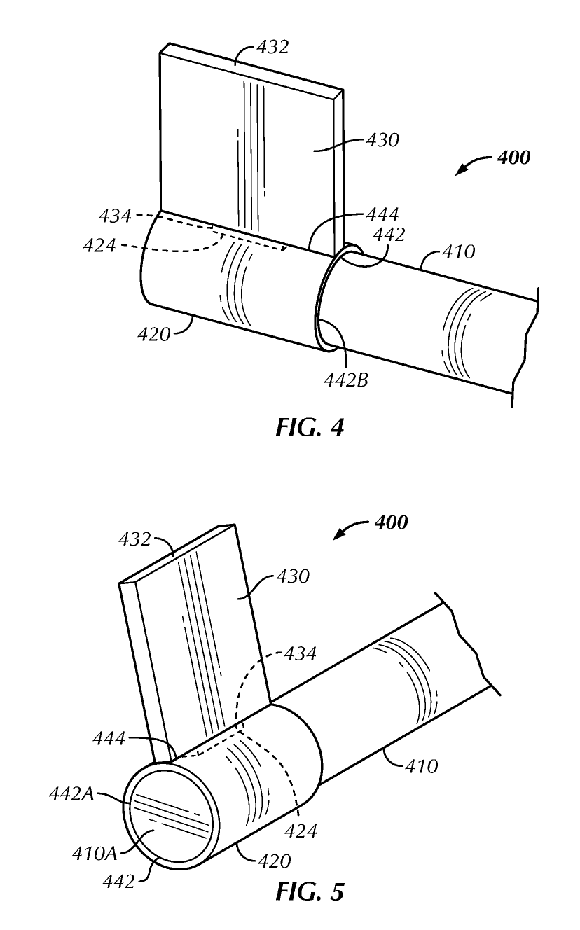

Referring now to FIGS. 4-7, the medical cutting device 400, in various examples, is similar in many respects to the medical cutting devices 100, 200 described herein. For instance, in some examples, the medical cutting device 400 includes the intermediate member 420 disposed proximate the distal end 410A of the elongate member 410. However, in some examples, the intermediate member 420 of the medical cutting device 400 includes a slotted hypotube instead of the block described with respect to the medical cutting device 100 and the hypotube described with respect to the medical cutting device 200. That is, in some examples, the intermediate member 420 is a hypotube that extends around and is attached to the elongate member 410 and includes at least one slot 424 within a sidewall of the intermediate member 420. In some examples, the at least one slot 424 extends completely through the sidewall of the intermediate member 420, such that the slot 424 extends from an exterior of the intermediate member 420 to a lumen 422 of the intermediate member 420. In other examples, the slot 424 can extend only partially through the sidewall of the intermediate member 420, thereby forming a channel in the sidewall.

In some examples, at least a portion of the cutting member 430 is disposed within the slot 424. In some examples, the cutting member 430 includes a tab 434 sized and shaped to fit at least partially within the slot 424. In some examples, the slot 424 can extend an entire length of the cutting member 430, such that an end of the cutting member 430 forms the tab 434 of the cutting member 430 for placement at least partially within the slot 424 of the intermediate member 420.

In some examples, the intermediate member 420 is attached to the elongate member 410 at a first joint 442. In further examples, the intermediate member 420 is welded to the elongate member 410 at the first joint 442. In further examples, the intermediate member 420 is laser welded to the elongate member 410 at the first joint 442. In some examples, the first material of the elongate member 410 is compatible with the second material of the intermediate member 420 so as to create a relatively strong weld at the first joint 442. As described above, in some examples, the first material includes a titanium alloy and the second material includes a cobalt alloy, which are compatible materials for forming a robust weld. In some examples, the first joint 442 is formed at least partially around a substantially circumferential abutment line 442A, 442B between the elongate member 410 and the intermediate member 420. In further examples, the first joint 442 is formed along an entirety of the circumferential abutment line 442A, 442B between the elongate member 410 and the intermediate member 420. In various examples, the first joint 442 can be formed at least partially along a distal circumferential abutment line 442A, at least partially along a proximal circumferential abutment line 442B, or at least partially along both the distal circumferential abutment line 442A and the proximal circumferential abutment line 442B.

With the tab 434 in place at least partially within the slot 424, in some examples, the cutting member 430 is attached to the intermediate member 420 at a second joint 444. In some examples, the tab 434 of the cutting member 430 is received within the slot 424 of the intermediate member 420 with the cutting member 430 attached to the intermediate member 420 at the second joint, which is spaced from an end of the tab 434. In further examples, the end of the tab 434 can contact the elongate member 410 with the tab 434 of the cutting member 430 disposed within the slot 424 of the intermediate member 420. The configuration of the cutting member 430 and the intermediate member 420, with the tab 434 at least partially received within the slot 424, helps the strength and stability of the attachment of the cutting member 430 to the intermediate member 420. In further examples, the cutting member 430 is welded to the intermediate member 420 at the second joint 444. In further examples, the cutting member 430 is laser welded to the intermediate member 420 at the second joint 444. In some examples, the third material of the cutting member 430 is compatible with the second material of the intermediate member 420 so as to create a relatively strong weld at the second joint 444. As described above, in some examples, the third material includes a ferrous alloy and the second material includes a cobalt alloy, which are compatible materials for forming a robust weld. In some examples, the second joint 444 is formed at least partially along an abutment line between the cutting member 430 and the intermediate member 420. In further examples, the second joint 444 is formed along an entirety of the abutment line between the cutting member 430 and the intermediate member 420. In some examples, the intermediate member 420 includes a hypotube with an exterior surface, wherein the cutting member 430 at least partially abuts the exterior surface of the intermediate member 420 for attachment to the intermediate member 420 at the second joint 444.

The cutting member 430, in some examples, includes a rectangular sheet extending outwardly from the intermediate member 420. In some examples, the cutting member 430 includes the sharpened edge 432 facing substantially sideways or radially with respect to the medical cutting device 400. In some examples, the cutting member 430 includes a distal sharpened edge (for instance, similar to the distal sharpened edge 132′ of the medical cutting device 100) facing substantially distally with respect to the medical cutting device 400 instead of the sharpened edge 432. In other examples, the cutting member 430 includes a proximal sharpened edge (for instance, similar to the proximal sharpened edge 132″ of the medical cutting device 100) facing substantially proximally with respect to the medical cutting device 400 instead of the sharpened edge 432. In still other examples, the cutting member 430 includes a combination of two or more of the sharpened edge 432, the distal sharpened edge, and the proximal sharpened edge.

In other examples, the cutting member can include a shape other than rectangular, as described above, with one or more sharpened edges, for instance, if a different shape would be more conducive to performing in a particular procedure. In some examples, the cutting member 430 extends from the intermediate member 420 in a radially outward direction from the elongate member 410. In other examples, the cutting member can extend outwardly from the intermediate member at an angle with a tangential component to the elongate member. In some examples, the cutting member 430 can be configured to fold down and/or at least partially wrap around the elongate member 410, for instance, to allow for the medical cutting device 400 to be loaded into and/or passed through a catheter, an introducer, or some other delivery device. In some examples, the cutting member 430 extends substantially longitudinally with respect to the elongate member 410. In other examples, the cutting member 430 can extends along a length of the elongate member 410 angled from longitudinal (for instance, at least partially spirally along the elongate member 410). In further examples, the cutting member 430 can extend from the intermediate member 420 at least partially circumferentially around the elongate member 410. In some examples, at least a portion of the cutting member 430 extends distally with respect to the elongate member 410. In further examples, the cutting member 430 at least partially extends distally away from the distal end 410A of the elongate member 410, in a manner similar to that which is described above with respect to the portion 230′ of the cutting member 230 of the medical cutting device 200, so as to precede the elongate member 410 and allow for the medical cutting device 400 to cut an object disposed immediately in front of the medical cutting device 400 in addition to or instead of cutting to the side of the medical cutting device 400.

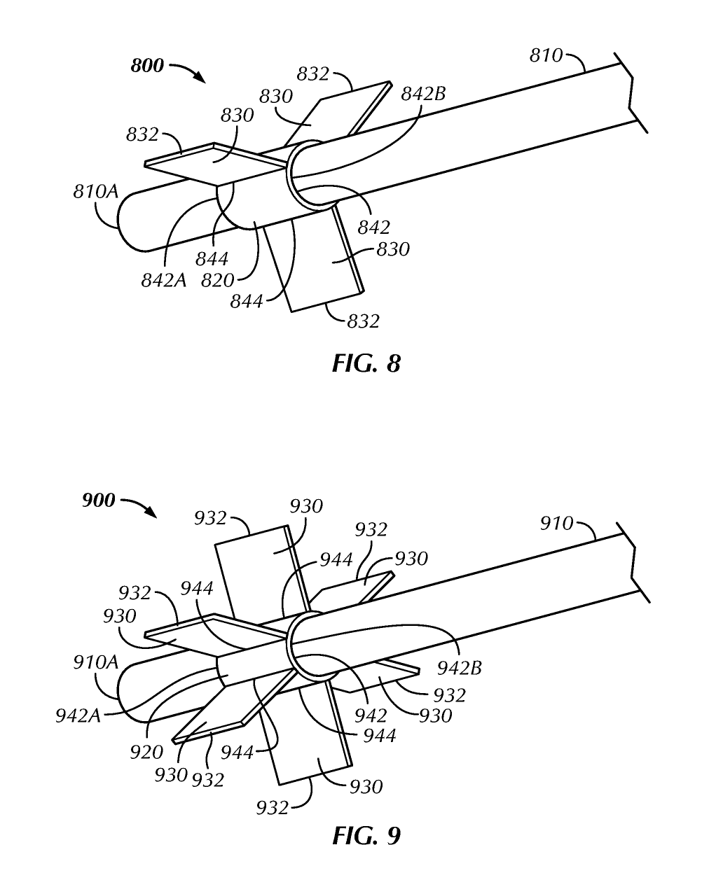

Referring now to FIG. 8, the medical cutting device 800, in various examples, is similar in many respects to the medical cutting devices 100, 200, 400 described herein. For instance, in some examples, the medical cutting device 800 includes the intermediate member 820 disposed proximate the distal end 810A of the elongate member 810. In some examples, the intermediate member 820 of the medical cutting device 800 includes a hypotube similar to the hypotube of the intermediate member 220 described herein with respect to the medical cutting device 200. In some examples, the intermediate member 820 of the medical cutting device 800 includes a slotted hypotube similar to the slotted hypotube of the intermediate member 420 described herein with respect to the medical cutting device 400. In the example shown in FIG. 8, the intermediate member 820 is positioned on the elongate member 810 at a location proximally spaced from the distal end 810A of the elongate member 810. However, in other examples, the intermediate member 820 can be disposed at the distal end 810A of the elongate member 810.

In some examples, the medical cutting device 800 includes more than one cutting member 830 extending outwardly from the intermediate member 820 and, in turn, the elongate member 810. In the example shown in FIG. 8, the medical cutting device 800 includes three cutting members 830. However, this is merely exemplary and is not intended to be limiting. In other examples, it is contemplated that the medical cutting device includes more or fewer than three cutting members 830, for instance, depending upon the procedure for which the medical cutting device 800 is to be used; the material, tissue, or other object to be cut with the medical cutting device 800; or the location within the patient to be accessed, to name a few. In some examples, the cutting members 830 are substantially evenly spaced from one another. In other examples, the cutting members 830 are unevenly spaced from one another.

In some examples, the intermediate member 820 is attached to the elongate member 810 at a first joint 842. In further examples, the intermediate member 820 is welded to the elongate member 810 at the first joint 842. In further examples, the intermediate member 820 is laser welded to the elongate member 810 at the first joint 842. In some examples, the first material of the elongate member 810 is compatible with the second material of the intermediate member 820 so as to create a relatively strong weld at the first joint 842. As described above, in some examples, the first material includes a titanium alloy and the second material includes a cobalt alloy, which are compatible materials for forming a robust weld. In some examples, the first joint 842 is formed at least partially around a substantially circumferential abutment line 842A, 842B between the elongate member 810 and the intermediate member 820. In further examples, the first joint 842 is formed along an entirety of the circumferential abutment line 842A, 842B between the elongate member 810 and the intermediate member 820. In various examples, the first joint 842 can be formed at least partially along a distal circumferential abutment line 842A, at least partially along a proximal circumferential abutment line 842B, or at least partially along both the distal circumferential abutment line 842A and the proximal circumferential abutment line 842B.

In some examples, each of the cutting members 830 is attached to the intermediate member 820 at a second joint 844. In further examples, each of the cutting members 830 is welded to the intermediate member 820 at the second joint 844. In further examples, each of the cutting members 830 is laser welded to the intermediate member 820 at the second joint 844. In some examples, the third material of the cutting members 830 is compatible with the second material of the intermediate member 820 so as to create a relatively strong weld at each of the second joints 844. As described above, in some examples, the third material includes a ferrous alloy and the second material includes a cobalt alloy, which are compatible materials for forming a robust weld. In some examples, each of the second joints 844 is formed at least partially along an abutment line between the respective cutting member 830 and the intermediate member 820. In further examples, each of the second joints 844 is formed along an entirety of the abutment line between the respective cutting member 830 and the intermediate member 820. In some examples, the intermediate member 820 includes a hypotube with an exterior surface, wherein each of the cutting members 830 abuts the exterior surface of the intermediate member 820 for attachment to the intermediate member 820 at the respective second joint 844.

The cutting members 830, in some examples, each includes a rectangular sheet extending outwardly from the intermediate member 820. In some examples, one or more of the cutting members 830 includes the sharpened edge 832 facing substantially sideways or radially with respect to the medical cutting device 800. In some examples, one or more of the cutting members 830 includes a distal sharpened edge (for instance, similar to the distal sharpened edge 132′ of the medical cutting device 100) facing substantially distally with respect to the medical cutting device 800 instead of the sharpened edge 832. In some examples, one or more of the cutting members 830 includes a proximal sharpened edge (for instance, similar to the proximal sharpened edge 132″ of the medical cutting device 100) facing substantially proximally with respect to the medical cutting device 800 instead of the sharpened edge 832. In some examples, one or more of the cutting members 830 includes a combination of two or more of the sharpened edge 832, the distal sharpened edge, and the proximal sharpened edge. In some examples, the cutting members 830 can include differently facing sharpened edges and/or combinations of sharpened edges from one another.

In other examples, the cutting member can include a shape other than rectangular, as described above, with one or more sharpened edges, for instance, if a different shape would be more conducive to performing in a particular procedure. In some examples, at least one of the cutting members of the medical cutting device can include a different shape from the other cutting members of the medical cutting device. In some examples, one or more of the cutting members 830 extend from the intermediate member 820 in a radially outward direction from the elongate member 810. In other examples, one or more of the cutting members can extend outwardly from the intermediate member at an angle with a tangential component to the elongate member. In some examples, one or more of the cutting members 830 can be configured to fold down and/or at least partially wrap around the elongate member 810, for instance, to allow for the medical cutting device 800 to be loaded into and/or passed through a catheter, an introducer, or some other delivery device. In some examples, one or more of the cutting members 830 extend substantially longitudinally with respect to the elongate member 810. In other examples, one or more of the cutting members 830 can extend along a length of the elongate member 810 angled from longitudinal (for instance, at least partially spirally along the elongate member 810). In further examples, one or more of the cutting members 830 can extend from the intermediate member 820 at least partially circumferentially around the elongate member 810. In some examples, at least a portion of one or more of the cutting members 830 extends distally with respect to the elongate member 810. In further examples, one or more of the cutting members 830 at least partially extend distally away from the distal end 810A of the elongate member 810, in a manner similar to that which is described above with respect to the portion 230′ of the cutting member 230 of the medical cutting device 200, so as to precede the elongate member 810 and allow for the medical cutting device 800 to cut an object disposed immediately in front of the medical cutting device 800 in addition to or instead of cutting to the side of the medical cutting device 800.

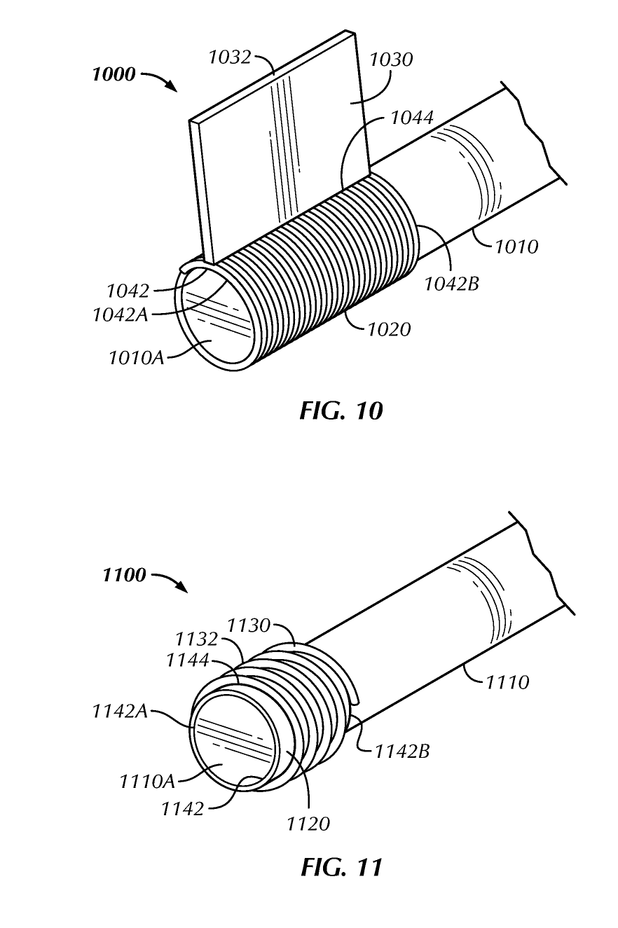

Referring now to FIG. 9, the medical cutting device 900, in various examples, is similar in many respects to the medical cutting devices 100, 200, 400, 800 described herein. For instance, in some examples, the medical cutting device 900 includes the intermediate member 920 disposed proximate the distal end 910A of the elongate member 910. In some examples, the intermediate member 920 of the medical cutting device 900 includes a hypotube similar to the hypotube of the intermediate member 220 described herein with respect to the medical cutting device 200. In some examples, the intermediate member 920 of the medical cutting device 900 includes a slotted hypotube similar to the slotted hypotube of the intermediate member 420 described herein with respect to the medical cutting device 400. In the example shown in FIG. 9, the intermediate member 920 is positioned on the elongate member 910 at a location proximally spaced from the distal end 910A of the elongate member 910. However, in other examples, the intermediate member 920 can be disposed at the distal end 910A of the elongate member 910.

In some examples, the medical cutting device 900 includes more than one cutting member 930 extending outwardly from the intermediate member 920 and, in turn, the elongate member 910. In the example shown in FIG. 9, the medical cutting device 900 includes six cutting members 930. However, this is merely exemplary and is not intended to be limiting. In other examples, it is contemplated that the medical cutting device includes more or fewer than six cutting members 930, for instance, depending upon the procedure for which the medical cutting device 900 is to be used; the material, tissue, or other object to be cut with the medical cutting device 900; or the location within the patient to be accessed, to name a few. In some examples, the cutting members 930 are substantially evenly spaced from one another. In other examples, the cutting members 930 are unevenly spaced from one another.

In some examples, the intermediate member 920 is attached to the elongate member 910 at a first joint 942. In further examples, the intermediate member 920 is welded to the elongate member 910 at the first joint 942. In further examples, the intermediate member 920 is laser welded to the elongate member 910 at the first joint 942. In some examples, the first material of the elongate member 910 is compatible with the second material of the intermediate member 920 so as to create a relatively strong weld at the first joint 942. As described above, in some examples, the first material includes a titanium alloy and the second material includes a cobalt alloy, which are compatible materials for forming a robust weld. In some examples, the first joint 942 is formed at least partially around a substantially circumferential abutment line 942A, 942B between the elongate member 910 and the intermediate member 920. In further examples, the first joint 942 is formed along an entirety of the circumferential abutment line 942A, 942B between the elongate member 910 and the intermediate member 920. In various examples, the first joint 942 can be formed at least partially along a distal circumferential abutment line 942A, at least partially along a proximal circumferential abutment line 942B, or at least partially along both the distal circumferential abutment line 942A and the proximal circumferential abutment line 942B.

In some examples, each of the cutting members 930 is attached to the intermediate member 920 at a second joint 944. In further examples, each of the cutting members 930 is welded to the intermediate member 920 at the second joint 944. In further examples, each of the cutting members 930 is laser welded to the intermediate member 920 at the second joint 944. In some examples, the third material of the cutting members 930 is compatible with the second material of the intermediate member 920 so as to create a relatively strong weld at each of the second joints 944. As described above, in some examples, the third material includes a ferrous alloy and the second material includes a cobalt alloy, which are compatible materials for forming a robust weld. In some examples, each of the second joints 944 is formed at least partially along an abutment line between the respective cutting member 930 and the intermediate member 920. In further examples, each of the second joints 944 is formed along an entirety of the abutment line between the respective cutting member 930 and the intermediate member 920. In some examples, the intermediate member 920 includes a hypotube with an exterior surface, wherein each of the cutting members 930 abuts the exterior surface of the intermediate member 920 for attachment to the intermediate member 920 at the respective second joint 944.

The cutting members 930, in some examples, each includes a rectangular sheet extending outwardly from the intermediate member 920. In some examples, one or more of the cutting members 930 includes the sharpened edge 932 facing substantially sideways or radially with respect to the medical cutting device 900. In some examples, one or more of the cutting members 930 includes a distal sharpened edge (for instance, similar to the distal sharpened edge 132′ of the medical cutting device 100) facing substantially distally with respect to the medical cutting device 900 instead of the sharpened edge 932. In some examples, one or more of the cutting members 930 includes a proximal sharpened edge (for instance, similar to the proximal sharpened edge 132″ of the medical cutting device 100) facing substantially proximally with respect to the medical cutting device 900 instead of the sharpened edge 932. In some examples, one or more of the cutting members 930 includes a combination of two or more of the sharpened edge 932, the distal sharpened edge, and the proximal sharpened edge. In some examples, the cutting members 930 can include differently facing sharpened edges and/or combinations of sharpened edges from one another.

In other examples, the cutting member can include a shape other than rectangular, as described above, with one or more sharpened edges, for instance, if a different shape would be more conducive to performing in a particular procedure. In some examples, at least one of the cutting members of the medical cutting device can include a different shape from the other cutting members of the medical cutting device. In some examples, one or more of the cutting members 930 extend from the intermediate member 920 in a radially outward direction from the elongate member 910. In other examples, one or more of the cutting members can extend outwardly from the intermediate member at an angle with a tangential component to the elongate member. In some examples, one or more of the cutting members 930 can be configured to fold down and/or at least partially wrap around the elongate member 910, for instance, to allow for the medical cutting device 900 to be loaded into and/or passed through a catheter, an introducer, or some other delivery device. In some examples, one or more of the cutting members 930 extend substantially longitudinally with respect to the elongate member 910. In other examples, one or more of the cutting members 930 can extend along a length of the elongate member 910 angled from longitudinal (for instance, at least partially spirally along the elongate member 910). In further examples, one or more of the cutting members 930 can extend from the intermediate member 920 at least partially circumferentially around the elongate member 910. In some examples, at least a portion of one or more of the cutting members 930 extends distally with respect to the elongate member 910. In further examples, one or more of the cutting members 930 at least partially extend distally away from the distal end 910A of the elongate member 910, in a manner similar to that which is described above with respect to the portion 230′ of the cutting member 230 of the medical cutting device 200, so as to precede the elongate member 910 and allow for the medical cutting device 900 to cut an object disposed immediately in front of the medical cutting device 900 in addition to or instead of cutting to the side of the medical cutting device 900.

Referring now to FIG. 10, the medical cutting device 1000, in various examples, is similar in many respects to the medical cutting devices 100, 200, 400, 800, 900 described herein. For instance, in some examples, the medical cutting device 1000 includes the intermediate member 1020 disposed proximate the distal end 1010A of the elongate member 1010. However, in some examples, the intermediate member 1020 of the medical cutting device 1000 includes a coil instead of the hypotube described with respect to the medical cutting device 200. That is, in some examples, the intermediate member 1020 is a coil that extends around and is attached to the elongate member 1010.

In some examples, the intermediate member 1020 is attached to the elongate member 1010 at a first joint 1042. In further examples, the intermediate member 1020 is welded to the elongate member 1010 at the first joint 1042. In further examples, the intermediate member 1020 is laser welded to the elongate member 1010 at the first joint 1042. In some examples, the first material of the elongate member 1010 is compatible with the second material of the intermediate member 1020 so as to create a relatively strong weld at the first joint 1042. As described above, in some examples, the first material includes a titanium alloy and the second material includes a cobalt alloy, which are compatible materials for forming a robust weld. In some examples, the first joint 1042 is formed at least partially around a substantially circumferential abutment line 1042A, 1042B between the elongate member 1010 and the intermediate member 1020. In further examples, the first joint 1042 is formed along an entirety of the circumferential abutment line 1042A, 1042B between the elongate member 1010 and the intermediate member 1020. In various examples, the first joint 1042 can be formed at least partially along a distal circumferential abutment line 1042A, at least partially along a proximal circumferential abutment line 1042B, or at least partially along both the distal circumferential abutment line 1042A and the proximal circumferential abutment line 1042B. In some examples, the intermediate member 1020 can be attached at one or more points between the distal circumferential abutment line 1042A and the proximal circumferential abutment line 1042B if the coil of the intermediate member 1020 includes space between loops of the coil.

In some examples, the cutting member 1030 is attached to the intermediate member 1020 at a second joint 1044. In further examples, the cutting member 1030 is welded to the intermediate member 1020 at the second joint 1044. In further examples, the cutting member 1030 is laser welded to the intermediate member 1020 at the second joint 1044. In some examples, the third material of the cutting member 1030 is compatible with the second material of the intermediate member 1020 so as to create a relatively strong weld at the second joint 1044. As described above, in some examples, the third material includes a ferrous alloy and the second material includes a cobalt alloy, which are compatible materials for forming a robust weld. In some examples, the second joint 1044 is formed at least partially along an abutment line between the cutting member 1030 and the intermediate member 1020. In further examples, the second joint 1044 is formed along an entirety of the abutment line between the cutting member 1030 and the intermediate member 1020. In some examples, the cutting member 1030 can be attached to the intermediate member 1020 at one or more locations where the cutting member 1030 abuts one or more of the loops of the coil of the intermediate member 1020.

The cutting member 1030, in some examples, includes a rectangular sheet extending outwardly from the intermediate member 1020. In some examples, the cutting member 1030 includes the sharpened edge 1032 facing substantially sideways or radially with respect to the medical cutting device 1000. In some examples, the cutting member 1030 includes a distal sharpened edge (for instance, similar to the distal sharpened edge 132′ of the medical cutting device 100) facing substantially distally with respect to the medical cutting device 1000 instead of the sharpened edge 1032. In other examples, the cutting member 1030 includes a proximal sharpened edge (for instance, similar to the proximal sharpened edge 132″ of the medical cutting device 100) facing substantially proximally with respect to the medical cutting device 1000 instead of the sharpened edge 1032. In still other examples, the cutting member 1030 includes a combination of two or more of the sharpened edge 1032, the distal sharpened edge, and the proximal sharpened edge.

In other examples, the cutting member can include a shape other than rectangular, as described above, with one or more sharpened edges, for instance, if a different shape would be more conducive to performing in a particular procedure. In some examples, the cutting member 1030 extends from the intermediate member 1020 in a radially outward direction from the elongate member 1010. In other examples, the cutting member can extend outwardly from the intermediate member at an angle with a tangential component to the elongate member. In some examples, the cutting member 1030 can be configured to fold down and/or at least partially wrap around the elongate member 1010, for instance, to allow for the medical cutting device 1000 to be loaded into and/or passed through a catheter, an introducer, or some other delivery device. In some examples, the cutting member 1030 extends substantially longitudinally with respect to the elongate member 1010. In other examples, the cutting member 1030 can extends along a length of the elongate member 1010 angled from longitudinal (for instance, at least partially spirally along the elongate member 1010). In further examples, the cutting member 1030 can extend from the intermediate member 1020 at least partially circumferentially around the elongate member 1010. In some examples, at least a portion of the cutting member 1030 extends distally with respect to the elongate member 1010. In further examples, the cutting member 1030 at least partially extends distally away from the distal end 1010A of the elongate member 1010, in a manner similar to that which is described above with respect to the portion 230′ of the cutting member 230 of the medical cutting device 200, so as to precede the elongate member 1010 and allow for the medical cutting device 1000 to cut an object disposed immediately in front of the medical cutting device 1000 in addition to or instead of cutting to the side of the medical cutting device 1000.

Referring now to FIG. 11, the medical cutting device 1100, in various examples, is similar in many respects to the medical cutting devices 100, 200, 400, 800, 900, 1000 described herein. For instance, in some examples, the medical cutting device 1100 includes the intermediate member 1120 disposed proximate the distal end 1110A of the elongate member 1110. In some examples, the intermediate member 1120 of the medical cutting device 1100 includes a hypotube similar to the hypotube described with respect to the medical cutting device 200. That is, in some examples, the intermediate member 1120 is a hypotube that extends around and is attached to the elongate member 1110.

In some examples, the intermediate member 1120 is attached to the elongate member 1110 at a first joint 1142. In further examples, the intermediate member 1120 is welded to the elongate member 1110 at the first joint 1142. In further examples, the intermediate member 1120 is laser welded to the elongate member 1110 at the first joint 1142. In some examples, the first material of the elongate member 1110 is compatible with the second material of the intermediate member 1120 so as to create a relatively strong weld at the first joint 1142. As described above, in some examples, the first material includes a titanium alloy and the second material includes a cobalt alloy, which are compatible materials for forming a robust weld. In some examples, the first joint 1142 is formed at least partially around a substantially circumferential abutment line 1142A, 1142B between the elongate member 1110 and the intermediate member 1120. In further examples, the first joint 1142 is formed along an entirety of the circumferential abutment line 1142A, 1142B between the elongate member 1110 and the intermediate member 1120. In various examples, the first joint 1142 can be formed at least partially along a distal circumferential abutment line 1142A, at least partially along a proximal circumferential abutment line 1142B, or at least partially along both the distal circumferential abutment line 1142A and the proximal circumferential abutment line 1142B.

In some examples, the cutting member 1130 is attached to the intermediate member 1120 at a second joint 1144. In further examples, the cutting member 1130 is welded to the intermediate member 1120 at the second joint 1144. In further examples, the cutting member 1130 is laser welded to the intermediate member 1120 at the second joint 1144. In some examples, the third material of the cutting member 1130 is compatible with the second material of the intermediate member 1120 so as to create a relatively strong weld at the second joint 1144. As described above, in some examples, the third material includes a ferrous alloy and the second material includes a cobalt alloy, which are compatible materials for forming a robust weld. In some examples, the second joint 1144 is formed at least partially along an abutment line between the cutting member 1130 and the intermediate member 1120. In further examples, the second joint 1144 is formed along an entirety of the abutment line between the cutting member 1130 and the intermediate member 1120.

The cutting member 1130, in some examples, includes a coil extending outwardly from and at least partially along the intermediate member 1120. The abrading surface 1132 of the cutting member 1130, in some examples, is configured to scrape away a surface of the object disposed within the patient. In various examples, the abrading surface 1132 includes, for instance, teeth, holes, grit, protrusions, scoops, or the like, or a combination thereof configured to abrade, wear down, or otherwise remove at least a portion of the material, tissue, or other object within the patient. In some examples, the cutting member 1130 extends from the intermediate member 1120 in a radially outward direction from the elongate member 1110. In some examples, the cutting member 1130 is sized or otherwise configured to allow for the medical cutting device 1100 to be loaded into and/or passed through a catheter, an introducer, or some other delivery device. In some examples, the cutting member 1130 at least partially extends distally away from the distal end 1110A of the elongate member 1110, in a manner similar to that which is described herein with respect to the medical cutting device 200, so as to precede the elongate member 1110 and allow for the medical cutting device 1100 to abrade, cut, or otherwise remove at least a portion of an object disposed immediately in front of the medical cutting device 1100 in addition to or instead of cutting to the side of the medical cutting device 1100.

The present inventors have recognized various advantages of the subject matter described herein. The present inventors have recognized, among other things, that the present subject matter can be used to perform a cutting or removal procedure within a patient, such as, for instance, within the vasculature of the patient. In various examples, the present subject matter is advantageous in that it provides an elongate member that is capable of being navigated within the patient (for instance, through vasculature of the patient) to a location within the patient where material is located that is to be cut, abraded, or otherwise removed from within the patient. To that end, the present inventors have recognized the present subject matter can allow for a medical device including the elongate member being at least partially formed from a material that allows navigation through the vasculature of the patient attached to a cutting member that can be formed, sharpened, or otherwise configured to allow for cutting, abrading, or otherwise removing material from within the patient. Also, the present subject matter is advantageous in that it provides for attachment of the elongate member, made from a first material, and the cutting member, made from a second material, even though, in some examples, the first and second materials are difficult to attach to one another. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A medical cutting device configured for at least partial insertion within a patient, the medical cutting device comprising:
a flexible elongate member including a distal end and a proximal end, the elongate member including a first material;
an intermediate member attached to the elongate member proximate the distal end of the elongate member, the intermediate member including a second material different from the first material; and
a cutting member attached to and extending outwardly from the intermediate member, the cutting member including a third material different from the first and second materials, wherein the cutting member is configured to cut an object disposed within the patient.

2. The medical cutting device of claim 1, wherein the first material of the elongate member includes a titanium alloy.

3. The medical cutting device of claim 1, wherein the second material of the intermediate member includes a cobalt alloy.

4. The medical cutting device of claim 1, wherein the third material of the cutting member includes a ferrous material.

5. The medical cutting device of claim 1, wherein the elongate member includes at least one of a wire, a hypotube, a cable, a braid, and a coil.

6. The medical cutting device of claim 1, wherein the intermediate member includes a hypotube disposed at least partially around the distal end of the elongate member.

7. The medical cutting device of claim 1, wherein the cutting member includes a sharpened edge configured to slice through the object disposed within the patient.

8. The medical cutting device of claim 1, wherein the cutting member includes an abrading surface configured to scrape away a surface of the object disposed within the patient.

9. The medical cutting device of claim 1, comprising more than one cutting member extending outwardly from the elongate member.

10. The medical cutting device of claim 1, wherein:
the intermediate member includes a slot; and
the cutting member includes a tab sized and shaped to fit within the slot, the tab being disposed within the slot.

11. A medical cutting device configured for at least partial insertion within a patient, the medical cutting device comprising:
a flexible elongate member including a distal end and a proximal end, the elongate member including a titanium alloy;
an intermediate member welded to the elongate member proximate the distal end of the elongate member, the intermediate member including a cobalt alloy; and
a cutting member welded to and extending outwardly from the intermediate member, the cutting member including a ferrous material, wherein the cutting member is configured to cut an object disposed within the patient.

12. The medical cutting device of claim 11, wherein the elongate member includes at least one of a wire, a hypotube, a cable, a braid, and a coil.

13. The medical cutting device of claim 11, wherein the intermediate member includes a hypotube disposed at least partially around the distal end of the elongate member.

14. The medical cutting device of claim 11, wherein the cutting member includes a sharpened edge configured to slice through the object disposed within the patient.

15. The medical cutting device of claim 11, wherein the cutting member includes an abrading surface configured to scrape away a surface of the object disposed within the patient.

16. The medical cutting device of claim 11, wherein:
the intermediate member includes a slot; and
the cutting member includes a tab sized and shaped to fit within the slot, the tab being disposed within the slot.

17. A medical cutting device configured for at least partial insertion within a patient, the medical cutting device comprising:
a flexible elongate member including a distal end and a proximal end, the elongate member including Nitinol;
an intermediate member laser welded to the elongate member proximate the distal end of the elongate member, the intermediate member including MP35N; and
a cutting member laser welded to and extending outwardly from the intermediate member, the cutting member including stainless steel, wherein the cutting member is configured to cut an object disposed within the patient.

18. The medical cutting device of claim 17, wherein the cutting member includes a sharpened edge configured to slice through the object disposed within the patient.

19. The medical cutting device of claim 17, wherein the cutting member includes an abrading surface configured to scrape away a surface of the object disposed within the patient.

20. The medical cutting device of claim 17, wherein:
the intermediate member includes a slot; and
the cutting member includes a tab sized and shaped to fit within the slot, the tab being disposed within the slot.
